(19) United States
(12) Patent Application Publication
(10) Pub. No.: US 2020/0069529 A1
(43) Pub. Date: Mar. 5, 2020

(54) Stoma Cleaning Kit

(71) Applicant: O&M Halyard, Inc., Mechanicsville, VA (US)

(72) Inventors: Kay B. Starnes, Alpharetta, GA (US); Donald McMichael, Roswell, GA (US); Khoa T. Lien, Alpharetta, GA (US)

(21) Appl. No.: 16/115,811

(22) Filed: Aug. 29, 2018

(51) Int. Cl. A61J 15/00; A45C 11/00; A45C 13/02; A61F 13/38; A61M 39/16; A61F 15/00; A61B 42/40; A61B 46/20

(52) U.S. Cl. CPC A61J 15/0015; A45C 11/008; A45C 13/02; A61J 15/0049; A45C 2011/007; A61M 39/16; A61F 15/001; A61B 42/40; A61B 46/20; A61F 13/38

(57) ABSTRACT

A kit for cleaning a stoma is generally provided including a container, a plurality of stoma cleaning accessories, and a first external access surface. The container includes a plurality of exterior surfaces housing a plurality of dividers defining separate compartments within the container. The plurality of exterior surfaces defines an opening such that the separate compartments are accessible by a user of the kit. Each of the separate compartments is configured to receive at least one of the plurality of stoma cleaning accessories. The first external access surface is operable with at least one of the exterior surfaces to selectably allow access to the separate compartments. At least one of the exterior surfaces or the first external access surface includes instructions and an indication of an order that each of the plurality of stoma cleaning accessories is utilized to clean the stoma such that each stoma cleaning accessory is sequentially utilized.

### FIELD OF THE INVENTION

The present invention relates to a cleaning kit used in conjunction with enteral feeding devices. More particularly, the present invention relates to a reusable cleaning kit dispenser for cleaning a stoma.

### BACKGROUND OF THE INVENTION

Numerous situations exist in which interior parts of the human body need to be catheterized through an artificial stoma to achieve a desired medical goal. Relatively common situations are for drainage of retained fluids and administering nutritional solutions or medicines directly into the stomach or intestines. For these situations a stoma is formed percutaneously and an indwelling device is placed through the stoma. By way of example, the surgical opening and/or the procedure to create a stoma spanning between the stomach or intestinal wall and the exterior of the skin is commonly referred to as gastrostomy, jejunostomy, ileostomy, and/or colostomy. A device with a catheter component, e.g., a feeding tube, placed through such a stoma allows injection of feeding solutions through the tube to provide nutrients directly to the stomach or intestines (known as enteral feeding). A variety of different devices intended for enteral feeding have been developed over the years, including some having a “low profile” relative to that portion which sits on a patient's skin, as well as those having the more traditional or non-low profile configuration. These percutaneous transconduit devices are frequently referred to as “gastrostomy tubes,” “percutaneous gastrostomy tubes,” “PEG tubes,” or “enteral feeding tubes.” These devices, which can also be referred to as feeding tubes, can have an external bolster that rests against the surface of the patient's skin and an internal bolster or balloon that rests against the stomach wall to hold the device in place at the stoma site.

One problem with the use of stomas and associated feeding tubes is proper care and cleaning. Often, a daily cleaning routine of the stoma is critical to promote stoma health, avoid infection, prevent skin redness, and avoid overall soreness of the stoma. Further, patients and caregivers are generally required to assemble the supplies for cleaning the stoma from independent sources. In addition, caregivers may find it difficult to understand how to properly clean the stoma by using the cleaning supplies from such independent sources.

Accordingly, an apparatus and associated method for cleaning stomas that reduces the complexity of the cleaning procedure while promoting a thorough cleaning would be welcome in the art.

### SUMMARY OF THE INVENTION

Aspects and advantages of the invention will be set forth in part in the following description, or may be obvious from the description, or may be learned through practice of the invention.

In one particular aspect, the present invention is directed to a kit for cleaning a stoma. The kit includes a container, a plurality of stoma cleaning accessories, and a first external access surface. The container includes a plurality of exterior surfaces housing a plurality of dividers such that the plurality of dividers defines separate compartments within the container. The plurality of exterior surfaces defines an opening such that the separate compartments are accessible by a user of the kit. Further, each of the separate compartments is configured to receive at least one of the plurality of stoma cleaning accessories. The first external access surface is operable with at least one of the exterior surfaces such that the first external access surface selectably allows access to the separate compartments. Additionally, at least one of the exterior surfaces or the first external access surface includes instructions and an indication of an order that each of the plurality of stoma cleaning accessories is utilized to clean the stoma. As such, each of the plurality of stoma cleaning accessories is sequentially utilized to clean the stoma.

In one embodiment, first external access surface may include a first hinged lid. In certain embodiments, the first hinged lid may include a pouch on an outward face of the first hinged lid. The pouch may define a cavity configured to receive at least one of the stoma cleaning accessories. For example, the cavity of the pouch may be configured to receive at least one drape. In another embodiment, the first hinged lid may include a plurality of lid dividers coupled to an inward face of the first hinged lid. In such an embodiment, the plurality of lid dividers may define at least one lid compartment configured to receive at least one of the plurality of stoma cleaning accessories.

In a further embodiment, the kit may further include a second external access surface operable with at least one of the exterior surfaces or the first external access surface such that the second external access surface selectively allows access to the separate compartments. For example, in one embodiment, the second external access surface may include a second hinged lid. The second hinged lid may be operable with at least one of the external surfaces or the first external access surface to selectively allow access to the separate compartments.

In another embodiment of the kit, each of the exterior surfaces and the first external access surface may be light impermeable such that the contents of the container are not externally visible. In one particular embodiment, the second external access surface may also be light impermeable. In an additional embodiment, each of the exterior surfaces and the first external access surface includes a fashionable design such that the purpose of the kit is not apparent. In a further embodiment, each of the separate compartments may be sequentially marked in an order an associated cleaning accessory for each of the separate compartments is utilized to clean the stoma. In such embodiments, at least one of the separate compartments sequentially marked may include a marker comprising at least one of an alpha-numerical symbol or color code symbol. In another embodiment, at least one of the exterior surfaces or the first external access surface may include a compliance check-off functionality structure such that the user marks a successful use of at least one of the plurality of stoma cleaning accessories. For example, the user may mark a successful use of each of the plurality of stoma cleaning accessories. As such, the compliance check-off functionality structure may include at least one of a plurality of stickers or perforated punches to mark the successful use of each of the plurality of stoma cleaning accessories.

In a further embodiment of the kit, each of the plurality of stoma cleaning accessories may include a defined supply, for example at least a thirty day supply, of each stoma cleaning accessory. In one embodiment, at least one of the plurality of stoma cleaning accessories may include one or more gloves, drapes, cleaning solutions, wipes operable with the one or more cleaning solutions, swabs, stoma pads, or travel packs. For example, the plurality of stoma cleaning accessories may each include one or more gloves, drapes, cleaning solutions, wipes operable with the one or more cleaning solutions, swabs, stoma pads, or travel packs. In one particular embodiment, the drape may include instructions and an indication of an order that each of the plurality of stoma cleaning accessories is utilized to clean the stoma. It should be further understood that the kit may further include any of the additional features as described herein and in any combination.

In another aspect, the present invention is directed to a method of cleaning a stoma utilizing a kit defining a plurality of separate compartments, each configured to receive a stoma cleaning accessory. The method includes retrieving gloves from a first compartment and applying the gloves to the hands of a user. The method further includes retrieving a drape from a second compartment and positioning the drape as a working surface. Another step includes retrieving a cleaning solution from a third compartment and applying the cleaning solution to the stoma and/or areas of the patient's skin surrounding the stoma. One step of the method includes retrieving a wipe from a fourth compartment and using the wipe to clean and/or dry the stoma and/or the areas of the patient's skin surrounding the stoma utilizing the cleaning solution. The method additionally includes retrieving a swab from a fifth compartment and using the swab to clean and/or dry the stoma and/or the areas of the patient's skin surrounding the stoma. A further step of the method includes retrieving a stoma pad from a sixth compartment and applying the stoma pad to a medical device operable with the stoma.

In one embodiment, the method may further include marking the completion of at least one step of the method on a compliance check-off functionality structure. For example, the user may mark the completion of each step of the method. It should be further understood that the method may further include any of the additional features as described herein.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 is a schematic perspective view of a feeding tube with which the stoma cleaning kit of the present invention can be utilized.

FIG. 2 illustrates one embodiment of the kit for cleaning a stoma according to aspects of the present disclosure, particularly illustrating a container and first external access surface of the kit.

FIG. 3 illustrates another embodiment of the kit in a closed position, particularly illustrating the kit where the first and second external access surfaces are closed to block an opening of the kit.

FIG. 4 illustrates another embodiment of the kit where the container houses a plurality of stoma cleaning accessories, particularly illustrating the kit where separate compartments are configured to receive at least one of a plurality of stoma cleaning accessories.

FIG. 5 depicts one embodiment of a method of cleaning a stoma according to aspects of the present disclosure, particularly depicting a method utilizing a kit defining a plurality of separate compartments configured to each receive a stoma cleaning accessory.

Repeat use of reference characters in the present specification and drawings is intended to represent same or analogous features or elements of the invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

Generally speaking, the present invention relates to a kit and associated method for using the kit to clean a stoma. A kit including separate compartments for stoma cleaning accessories, as well as instructions for sequentially using the cleaning accessories, may allow for more thorough and consistent stoma cleanings. For example, stoma cleaning accessories purchased separately may not include instructions properly explaining how to use the stoma cleaning accessories together or may not be intended for cleaning a stoma at all. The separate compartments may be sequentially marked (e.g., with markers) in an associated order the cleaning accessories are intended to be used, thereby promoting a specific order and flow for cleaning the stoma. As such, the kit may help to insure proper procedures are used to clean the stoma. Further, the kit may assist users and/or patients in purchasing the proper medical supplies to use for cleaning the stoma. Such a kit may also provide a private location to store stoma cleaning accessories. In certain embodiments of the kit, a compliance check-off functionality structure may allow the user and/or patient to ensure daily stoma cleanings have been performed and provide a record of such stoma cleanings.

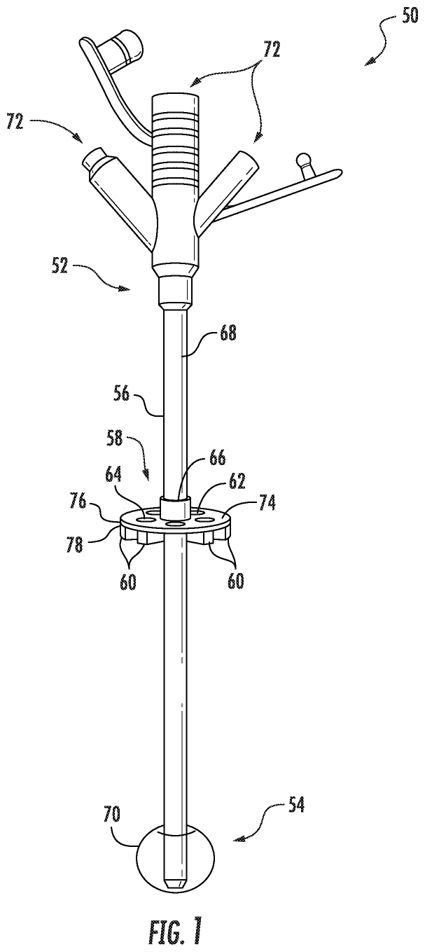

Referring now to FIG. 1 of the drawings, a schematic perspective view of a feeding tube 50 operable with the stoma and/or the stoma cleaning kit of the present invention. Though, it should be recognized that other feeding tubes with other configurations may be utilized with the presently disclosed stoma cleaning kit. Still, in other embodiments, the stoma cleaning kit of the present invention may be utilized with other medical devices and stomas operable with other medical devices.

The feeding tube 50 includes an external end 52 and an internal end 54 between which a tube 56 extends, where the tube 56 has a lumen 68 for delivering food, medication, or both to a patient via one or more ports 72. The external end 52 includes an external bolster 58 that can be positioned adjacent to a surface of a patient's skin, while the internal end 54 includes a retention balloon 70 that can be positioned adjacent to the patient's stomach wall. The external bolster 58 and retention balloon 70 hold the feeding tube 50 in place in a tract formed at the stoma site. The external bolster 58 can include a base 74 having an upper surface 76 and a lower surface 78. A sleeve 62 can be positioned against the upper surface 76 to receive the tube 56, and the base 74 can include a bore or opening 66 through which the tube 56 can pass, where the tube 56 enters the patient's stomach by passing through the abdominal wall and the stomach wall. The base 74 can also include air holes 64 that extend from the upper surface 76 to the lower surface 78 of the base 74, where the air holes 64 facilitate air flow around a stoma site. Although not required, multiple feet 60 can extend from the lower surface 78 of the base 74 of the external bolster 58, where the feet 60 can prevent the base 74 from contacting the stoma site and minimize the amount of contact between the surface of the patient's skin and the external bolster 58, which can improve aeration of the stoma site. In certain situations, fluid can leak from the stoma site, which can lead to skin infections and poor stoma health in general. As such, the stoma cleaning kit of the present invention can be used to clean any leakage from the stoma site as well as the feeding tube 50 itself if so desired.

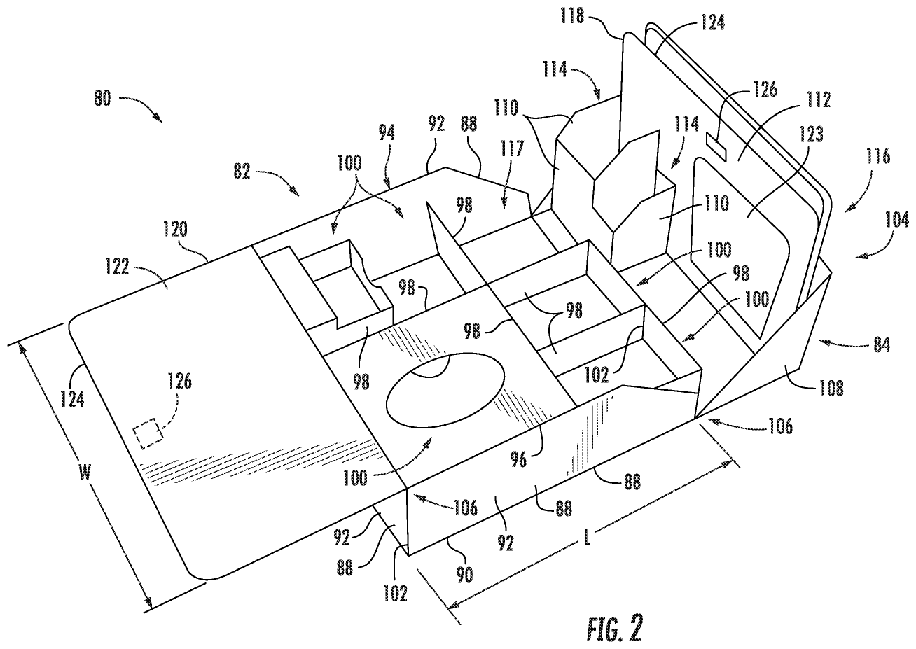

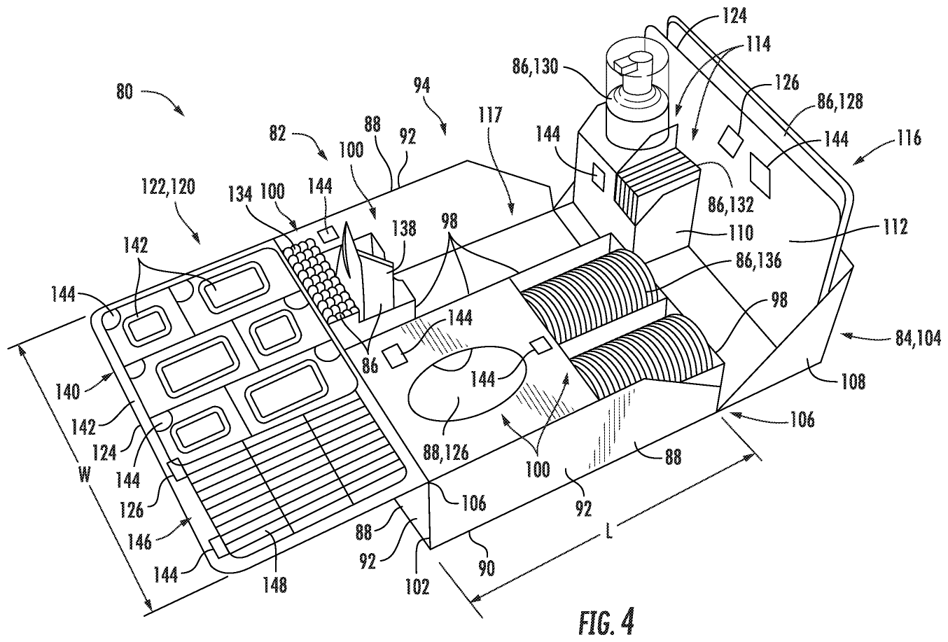

Referring now to FIG. 2, one embodiment of the kit 80 for cleaning a stoma is illustrated according to aspects of the present disclosure. Particularly, FIG. 2 illustrates a container 82 and a first external access surface 84 of the kit 100. The kit 80 may include the container 82, a plurality of stoma cleaning accessories 86 (see, e.g., FIG. 4), and the first external access surface 84. The container 82 may include a plurality of exterior surfaces 88. The exterior surfaces 88 may form a box. For instance, the plurality of exterior surfaces 88 may define a bottom 90 and one or more sides 92. In certain embodiments, the box may be a rectangle or a square. In further embodiments, the container 82 may include any shape defined by the plurality of exterior surfaces 88. For instance, the container 82 may be a triangle, circle, or other shape including a combination of one or more straight, curved, arced, linear, or non-linear exterior surfaces 88. Further, the container 82 may include more or less exterior surfaces 88, e.g., three or less or five or more exterior surfaces 88.

The plurality of exterior surfaces 88 may define an opening 94 such that an interior of the container 82 is accessible to a user of the kit 80, e.g., to access separate compartments 100. For example, a top 96 of the container 82 may not include a fixed, exterior surface 88. In the embodiment depicted, the opening 94 is positioned at the top 96 of the container 82 to allow access from the top 96. In further embodiments, it should be recognized that the opening 94 may be positioned anywhere on the container 82, such as one or more sides 82 and/or the bottom 90.

The container 82 may house a plurality of dividers 98 such that the plurality of dividers 98 defines separate compartments 100 within the container 82. For example, one or more straight dividers 98 may be extend between one exterior surface 88 and an opposite exterior surface 88 of the container 82. In one embodiment, a single divider 88 may extend across an entire length L of the container 82. In a further embodiment, a plurality of dividers 98 may be arranged end to end such that the dividers 98 extend the length L of the container 82. In still other embodiments, at least one of the dividers 98 may extend partially along the length L of container 82. In certain embodiments, one or more dividers 98 may include a notch such that further dividers 98 may be arranged approximately perpendicular to the divider 98 spanning the length L. For instance, a divider(s) 98 may extend at least partially along a width W of the container 82. In one embodiment, one or more dividers 98 may extend along the full width W of the container 82. Each of the dividers 98 may be coupled to at least another divider 98 or an exterior surface 88. In certain embodiments, dividers 98 extending along the length L or partially along the length L of the container 82 and dividers 98 extending along the width W or partially along the width W of the container 82 may define a grid of separate compartments 100.

It should be recognized that the divider(s) 98 may be coupled to other divider(s) 98 and/or the exterior surface(s) 88 using any suitable means, such as by adhesives, tape, welding, and/or mechanical fasteners (e.g., bolts, screws, and rivets). In one particular embodiment, the dividers 98 may be coupled using a tab notch interface operable with at least one other divider 98 or exterior surface 88. Further, the exterior surfaces 88 may be coupled together using any suitable means. It should be recognized that one or more of the exterior surfaces 88 or dividers 98 may be formed integrally together. For example, the exterior surfaces 88 and/or dividers 98 may be bent at joints 102 to define separate portions of single components (e.g., a first exterior surface 88, a second exterior surface 88, a first divider 98, or a second divider 98) formed from a solitary material. In certain embodiments, dividers 98 may be assembled together to form one or more of the separate compartments 100. In such embodiments, the separate compartment 100 may then be coupled to at least one of the exterior surfaces 88, such as at the bottom 90 and/or one or more sides 92. For instance, the separate compartment(s) 100 formed from the dividers 98 may be adhered to at least one of the exterior surfaces 88.

In certain embodiments, one or more dividers 98 may define a non-linear shape. For example, one or more dividers 98 may be curved, arced, or defined by any polynomial of any degree. In certain embodiments, one or more dividers 98 may define a joint 102 between two or more faces of a compartment 100. It should be recognized that the dividers 98 may be assembled to form any desirable shape. For example, the dividers 98 may be arranged in an order to form a compartment 100 with a shape corresponding to an associated cleaning accessory 86 (see, e.g., FIG. 4). Further, the kit 80 may define more or less separate compartments 100 as needed or desired for storing stoma cleaning accessories 86.

It should be recognized that the exterior surfaces 88, first external access surface 84, and/or dividers 98 may be formed from any suitable material or combination of materials. Further, a portion of the exterior surfaces 88, first external access surface 84, and/or dividers 98 may be formed from one material while another portion of the exterior surfaces 88, first external access surface 84, and/or dividers 98 may be formed from another material. Contemplated materials include, but are not limited to, cardboard, wood, plastic, polymers, composites, metals, or any combination thereof.

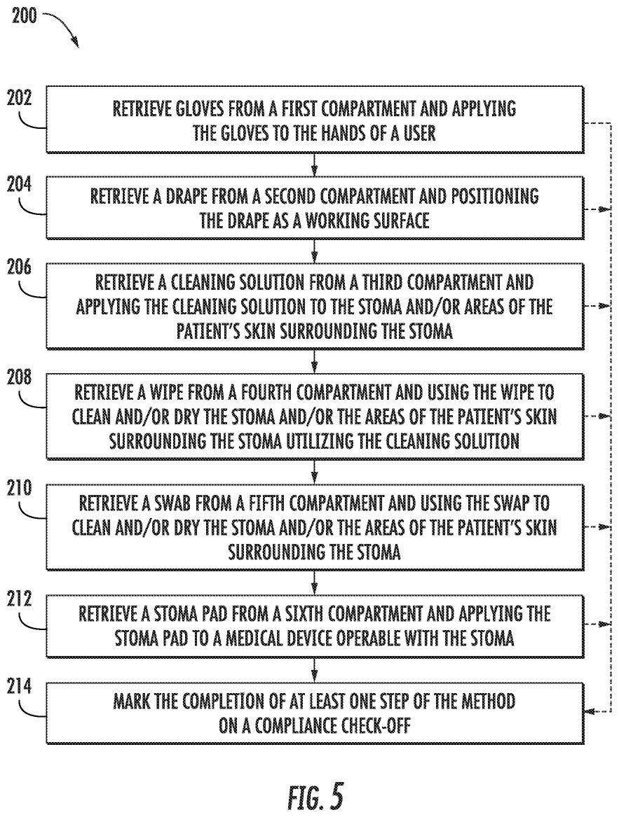

Further, each of the separate compartments 100 may be configured to receive at least one of the plurality of stoma cleaning accessories 86, as described in more detail below in regards to FIG. 4. As such, each of the plurality of stoma cleaning accessories 86 is sequentially utilized to clean the stoma, as will be described below in regards to FIGS. 4 and 5. More particularly, the kit 100 may include instructions 140 and associated markers 142 (e.g., alpha-numerical symbols, or colored symbols) that denote the order and flow each of the stoma cleaning accessories 86 is used to clean the stoma and/or medical device.

The first external access surface 84 may be operable with at least one of the exterior surfaces 88 such that the first external access surface 84 selectably allows access to the separate compartments 100. For instance the first external access surface 84 may selectively open or close the opening 94 of the container 82 to provide access inside the container 82. In one embodiment, the first external access surface 84 may be a seperatable lid configured to attach to one more of the exterior surfaces 88. For example, a tab-notch interface may be used to secure the lid. In other embodiments, the first external access surface 84 may be attached using any other suitable means. For instance, the lid may be attached using a tab-notch interface, a female-male interface, ties, a hook and loop fastener, or other means. In one embodiment, a weight of the first external access surface 84 may keep the first external access surface 84 secured to at least one of the exterior surfaces 88, and thus close the container 82.

In one embodiment, the first external access surface 84 may include a first hinged lid 104. For example, the first hinged lid 104 may be attached to one or more of the exterior surfaces 88 and/or dividers 98 at a pivot point 106. For instance, the pivot point 106 may extend along the full length L or width W. As shown, the pivot point 106 may be positioned at the bottom 90 of the container 82. As such, the first hinged lid 104 may also define at least one of the sides 92 of the container 82. In another embodiment, the first hinged lid 104 may be attached at a top 96 of the container 82. Further, the first hinged lid 104 may include one or more wings 108 used to secure the first hinged lid 104. As such, the wing(s) 108 may be operable with one or more exterior surfaces 88 defining the sides 92. For example, the wing(s) 108 may reduce displacements of the first hinged lid 104 along an axis of the pivot point 106 when the first hinged lid 104 is in a closed position, such as, for the embodiment depicted, along the width W.

In one embodiment, the first hinged lid 104 may include a plurality of lid dividers 110 coupled to an inward face 112 of the first hinged lid 104. In such an embodiment, the plurality of lid dividers 110 may define at least one lid compartment 114 configured to receive at least one of the plurality of stoma cleaning accessories 86, as described in more detail below in regards to FIG. 4. In such embodiments, the container 82 may define a pocket 117 configured to receive the lid compartment(s) 114. For example, the dividers 98 may be configured to leave the pocket 117 defining a void where the lid compartment(s) 114 will be positioned when the first hinged lid 104 is in a closed position. As such, the pocket 117 may allow for the container 82 to close without the lid compartment(s) 114, separate compartments 100, and/or stoma cleaning accessories 86 interfering with one another.

In certain embodiments, the first hinged lid 104 may include a pouch 116 on an outward face 118 of the first hinged lid 104. The pouch 16 may be coupled to the outward face 118 of the first hinged lid 104, or, in other embodiments, the pouch 116 may be formed integrally with the outward face 118 of the first hinged lid 104. The pouch 116 may define a cavity configured to receive at least one of the stoma cleaning accessories 86. Though, in other embodiments, it should be recognized the pouch may be coupled to the inward face 112 of the first hinged lid 104.

In a further embodiment, the kit 80 may further include a second external access surface 120 operable with at least one of the exterior surfaces 88 or the first external access surface 84 such that the second external access surface 120 selectively allows access to the separate compartments 100. For instance, the second external access surface 120 may selectively at least partially allow access to the interior of the compartment 82 via the opening 94. In one embodiment, the second external access surface 120 may include a second hinged lid 122. As shown, the second hinged lid 122 may be attached to one or more of the exterior surfaces 88 and/or dividers 98 at a pivot point 106. As shown, the pivot point 106 may be at the top 96 of the container 82. In other embodiments, it should be recognized that the second hinged lid 122 may generally be configured as the first hinged lid 104. For example, the second hinged lid 122 may be coupled to the container 82 at the bottom 90, include the lid divider(s) 110 defining the lid compartment(s) 114, include the pouch 116 on the inward face 112 and/or the outward face 118, and/or include one or more wings 108.

The second hinged lid 122 may be operable with at least one of the exterior surfaces 88 or the first external access surface 84 to selectively allow access to the separate compartments 100. For example, the second hinged lid 122 may be operable with at least the first hinged lid 104 to allow access to the separate compartments 100. In the depicted embodiment, the second hinged lid 122 closes before the first hinged lid 104. As such, the first hinged lid 104 at least partially secures the second hinged lid 122. In other embodiments, the kit 100 may be configured such that the first hinged lid 104 closes before the second hinged lid 122, at least partially securing the first hinged lid 104. In still other embodiments, the hinged lids 104, 122 may each close a portion of the container 82 and intersect at some point along the length L and/or the width W of the container 82. For example, the hinged lids 104, 122 may meet at approximately a center of the length L. In such an embodiment, one of the hinged lids 104, 122 may overlap the other of the hinged lids 104, 122. For instance, in the embodiment depicted, the first hinged lid 104 may overlap the second hinged lid 122 when the kit 80 is in a closed position. In other embodiments, the hinged lids 104, 122 may meet at approximately their respective edges 124 such that neither hinged lid 104, 122 overlaps the other. As such, the two hinged lids 104, 122 may be approximately as long as the length L of the container 82 when arranged in an end-to-end fashion (e.g., such as when the container 82 is in a closed position). Further, one or both of the hinged lids 104, 122 may include a closing mechanism 126 operable with the other hinged lid 104, 122 to securely close the container 82 (e.g., a latch, lock, tie, adhesive, hook and loop fastener, etc.).

In certain embodiments, the kit 80 may include a mirror 123 either permanently or selectively coupled to at least one of the exterior surfaces 88, the first external access surface 84 or the second external access surface 120. As such, the mirror 123 may assist users of the kit to perform the cleaning procedure using the stoma cleaning accessories 86 (see, e.g., FIGS. 4 and 5). More particularly, the mirror 123 may allow the patient to monitor the cleaning process, especially if the patient has difficulty seeing the stoma site.

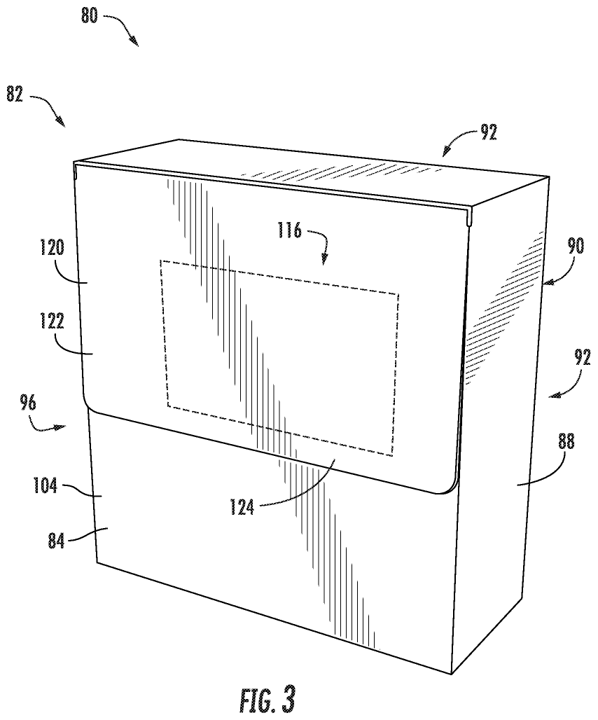

Referring now to FIG. 3, one embodiment of the kit 80 is illustrated in a closed position. More particularly, FIG. 3 illustrates the kit 80 where the first and second external access surfaces 84, 120 are closed to block the opening 94. For example the first and second hinged lids 104, 122 may be closed to prevent access to the interior of the container 82. Further, it should be recognized that the second hinged lid 122 closed over the first hinged lid 104 may secure the pouch 116 from external access and/or prevent stoma cleaning accessories 86 stored in the pouch 116 from falling out of the pouch 116. Though, in other embodiments, the second hinged lid 122 may only partially secure the pouch 116. For example, the pouch 116 may partially extend past the edge 124 of the second hinged lid 122. Still further, the pouch 116 may be fully visible in the closed position. For instance, the pouch 116 may be positioned past the edge 124 such that the second hinged lid 122 does not cover and/or block the pouch 116. Though, in other embodiments, the pouch 116 may be secured to an inward face 112 of the second hinged lid 122 to secure the pouch when the lids 104, 122 are closed.

In one embodiment of the kit 80, each of the exterior surfaces 88 and/or the first external access surface 84 may be light impermeable such that the contents of the container 82 are not externally visible. In one particular embodiment, the second external access surface 120 may also be light impermeable. It should be recognized that users of the kit 80 and/or patients may want to keep the contents of the kit 80 private. As such, an opaque stoma cleaning kit 80 may help insure the users' and/or patients' privacy while storing and/or transporting the stoma cleaning accessory(ies) 86. In additional embodiments, each of the exterior surfaces 88, the first external access surface 84, and/or the second external access surface 120 may include a fashionable design such that the purpose of the kit is not apparent. For instance, the outside of the kit 80 may be void of branding and/or logos that may indicate the purpose of the kit 80. Moreover, the kit 80 may be configured such that, when closed, the purpose of the contents is not obvious and designs and colors are used to assist the user in maintain personal discretion.

Referring now to FIG. 4, one embodiment of the kit 80 is illustrated where the container 82 houses a plurality of stoma cleaning accessories 86. The kit 80 of FIG. 4 may generally be configured the same as or similar to the kit 80 of FIG. 2. For instance, the kit 80 may include one or more the compartments 110, 114 and the pouch 116. Though omitted for clarity, the kit 80 of FIG. 4 may also include the mirror 123. More particularly, FIG. 4 illustrates the kit 80 where each of the separate compartments 100 may be configured to receive at least one of the plurality of stoma cleaning accessories 86. Further, the lid compartment 114 and the pouch 116 may each be configured to receive at least one of the plurality of stoma cleaning accessories 86. In one embodiment, at least one of the plurality of stoma cleaning accessories 86 may include one or more gloves, drapes, cleaning solutions, wipes operable with the one or more cleaning solutions, swabs, stoma pads, or travel packs. Though in other embodiments, more or less stoma cleaning accessories 86 may be utilized with the kit 80. Further, the kit 80 may not include some of the exemplary cleaning accessories 86 while including other stoma cleaning accessories 86. In one particular embodiment, the plurality of stoma cleaning accessories 86 may each include one or more gloves, drapes, cleaning solutions, wipes operable with the one or more cleaning solutions, swabs, stoma pads, or travel packs. Further, the cavity of the pouch 116 may be configured to receive at least one drape.

Each of the plurality of stoma cleaning accessories 86 may be used for a separate purpose and is intended to be used in a particular sequential order to clean the stoma. For example, the gloves 126 may help prevent the user from inadvertently exposing the stoma to bacteria, dirt, debris, infections, etc. during the cleaning procedure. Similarly, the drape(s) 128 may provide a relatively clean and dry surface to place other stoma cleaning accessories 86 while performing the cleaning procedure. As such, a disposable drape 128 may reduce the risk of infection to the stoma. Further, the drape(s) 128 may provide a convenient receptacle to place used stoma cleaning accessories 86 during the cleaning procedure. For example, the drape(s) 128 may define an internal cavity to receive used stoma cleaning accessories 86 such that all of the disposable, stoma cleaning accessories 86 may be easily discarded after the cleaning procedure is completed, such as the cleaning procedure represented by the method of FIG. 5.

The cleaning solution 130 may disinfect and remove dirt, debris, bodily fluids/substances, and/or foreign substances from the stoma. The cleaning solution 130 may include water and one or more detergents and/or disinfectants. The wipe(s) 132 may be operable with the cleaning solution 130 and assist in cleaning/disinfecting the stoma. The wipe(s) 132 may include known medical wipes, gauze, or similar substance suitable for cleaning the stoma. The swabs 134 also may be operable with the cleaning solution 130 to assist in cleaning/disinfecting the stoma. For instance, the swabs 134 may be used to clean small areas inaccessible to, or hard to properly clean with, the wipes 132. It should be recognized that the cleaning solution 130, wipes 132, and/or swabs 134 may also be used to clean a medical device operable with the stoma. For instance, the stoma cleaning accessories 86 may be used to clean the feeding tube 50 of FIG. 1. As an example, the swabs 134 may be used to clean the area of the medical device in contact with the skin or stoma. Further, in other embodiments, the medical device may be cleaned with other stoma cleaning accessories 86 and/or with additional stoma cleaning accessories 86. It should be recognized such additional stoma cleaning accessories 86 may be stored in the kit 80 and/or may be stored separately from the stoma cleaning accessories 86 meant to clean the stoma itself.

Further, the stoma pad(s) 136 may be applied to a medical device, such as the feeding tube 50 (see, e.g., FIG. 1), in order to help keep the stoma clean and dry. For instance, the stoma pad(s) 136 may include a cavity configured to receive external bolster 58 of the feeding tube 50. Further, the stoma pad(s) 136 may include a wicking layer arranged to be below the lower surface 78 of the feeding tube 50 at least partially between the feeding tube 50 and the patient's skin when the feeding tube 50 is inserted in the stoma. The wicking layer may wick moisture from the stoma and/or the patients skin surrounding the stoma. By removing moisture from the stoma, infection, redness, and soreness of the stoma site may be reduced or avoided. The stoma pad(s) 136 may also include an absorbent layer arranged to be on top of the upper surface 76 when the feeding tube 50 is inserted in the stoma. Such an absorbent layer may contain the moisture removed from the stoma site by the wicking layer. It should be recognized that, in other embodiments, the kit 80 may include stoma pad(s) 136 operable with other medical devices or other configurations of the feeding tube 50. For instance, the stoma pad(s) 50 may include more or less components and may not include the wicking layer, absorbent layer, and/or the cavity configured to receive the external bolster 58.

The travel pack(s) 138 may include a single use supply of the gloves 126, wipes 132 (either dry or including a cleaning solution 130), a separate cleaning solution 130, swabs 134, and/or a stoma pad 136. Such travel pack(s) 138 may be supplied for users and/or patients to conveniently travel with when a large kit would be difficult, inconvenient, and/or impossible to transport.

In a further embodiment of the kit 80, each of the plurality of stoma cleaning accessories 86 may include a defined supply, for example at least a thirty day supply, of each stoma cleaning accessory 86. For instance, some users may purchase a subscription that sends a thirty day supply of each stoma cleaning accessory 86 at predefined intervals, such as monthly. Though, in other embodiments, the kit 80 may include a supply of stoma cleaning accessories 86 for any desirable length of time, such as more than thirty days, such as two months, or such as three months or more. In other embodiments, kit 80 may include less than a thirty day supply of each stoma cleaning accessory 86. It should be recognized that the container 82, the separate compartments 100, the lid compartment 114, and/or the pouch 116 may be sized to approximately contain the desired supply of stoma cleaning accessories 86 for different periods of time. For instance, separate kits 80 may be configured to supply stoma accessories 86. As such, kits 80 may define separate sizes (e.g. small, medium, or large). Further, it should also be recognized that the kit 80 may include less of the travel packs 138 than other stoma cleaning accessories 86. For example, several travel packs 138 may constitute a thirty day supply of travel packs 138.

Still referring to FIG. 4, at least one of the exterior surfaces 88, the first external access surface 84, or the second external access surface 120 may include instructions 140 and an indication of an order that each of the plurality of stoma cleaning accessories 86 is utilized to clean the stoma. For example, in the depicted embodiment, the instructions 140 may be included on an inward face 142 of the second external access surface 120. As such, each of the plurality of stoma cleaning accessories 86 may be sequentially utilized to clean the stoma. It should be recognized that the instructions 140 may include written text and/or graphical representations 142 depicting how to use the stoma cleaning accessories 86 and the order the stoma cleaning accessories 86 should be used in. It also should be recognized that instructions 140 including graphical representations 142 may allow users that speak various languages to use the kit 80 without the need to translate the instructions 140. In one particular embodiment, the drape 128 may further include instructions 80 and an indication of an order that each of the plurality of stoma cleaning accessories 86 is utilized to clean the stoma.

In a further embodiment, each of the separate compartments 100, the lid compartment 114, and/or the pouch 116 may be sequentially marked in an order an associated stoma cleaning accessory 86 for each of the compartments 100, 114 and/or pouch 116 is utilized to clean the stoma. For instance, one or more of the separate compartments 100, 114 or the pouch 116 may include a marker 144 coupled to one or more dividers 98, exterior surfaces 88, lid dividers 110, the first external access surface 84, and/or the second external access surface 120 adjacent to each of the compartments 100, 114 and/or the pouch 116. For example, the compartments 110, 114 and/or the pouch 116 may be sequentially marked using alpha numerical symbols (e.g., “1, 2, 3” or “A, B, C”). In other embodiments, the marker(s) 144 for the compartments 100, 114 and/or the pouch 116 may be color coded. In still further embodiments, the marker(s) 144 may include any symbol that may be used to denote the order that each stoma cleaning accessory 86 is used. The instructions 140 may be linked to the markers 144 to assist in determining an order and flow of using the stoma cleaning accessories 86. As such, the instructions 140 may include the marker(s) 142 for each step of the instructions 140 corresponding to the marker(s) 142 associated with each of the stoma cleaning accessories 86. For instance, the markers 142 of the instructions 140 may use the corresponding color for the markers 142 of each compartment 100, 114 and/or the pouch 116 to order the use of the stoma cleaning accessories 86. It should be recognized that certain compartments 100, 114 and associated cleaning accessories 86 may share one alpha numerical symbol and/or color designation. For example, cleaning accessories 86 configured to be used together may share a single mark 142 designation to place their use in the order of the cleaning procedure (e.g., the cleaning solution 130, wipes 132, and/or swabs 134 may include a single alpha-numerical identifier and/or color identifier).

In another embodiment, at least one of the exterior surfaces 88, the first external access surface 84, or the second external access surface 120 may include a compliance check-off functionality structure 146 such that the user marks a successful use of at least one of the plurality of stoma cleaning accessories 86. For example, the user may mark a successful use of each of the plurality of stoma cleaning accessories 86. In certain embodiments, the user may mark a location(s) 148 corresponding to the use of an associated stoma cleaning accessory 86 for each day (e.g., with a pen or pencil). In certain embodiments, the user may mark a location 148 corresponding to completing an entire cleaning procedure for each day. In other embodiments, the compliance check-off 146 may include at least one of a plurality of stickers or perforated punches to mark the locations 148 after successful use of at least one of the plurality of stoma cleaning accessories 86. In certain other embodiments, the compliance check-off functionality structure 146 may include a dry-erase portion, or similar, that may allow the user to clear the marked successful uses of the kit 80 and associated stoma cleaning accessories 86, such as at the end of a thirty day supply of stoma cleaning accessories 86.

In at least one embodiment, the compliance check-off functionality structure 146 may be replaceable. For example, a new compliance check-off functionality structure 146 may be provided with each thirty day supply of stoma cleaning accessories 86, such as with each shipment of a subscription that delivers thirty day supplies of the stoma cleaning accessories 86. Though it should be recognized the period of time included on the compliance check-off functionality structure 146 may be any other suitable period of time. For example, the compliance check-off functionality structure 146 may include markable locations 148 corresponding to a week, two weeks, six weeks, two months, sixty days, three months, ninety days, or any other suitable period of time.

Referring now to FIG. 5, one embodiment of a method (200) of cleaning a stoma is depicted according to aspects of the present disclosure. Particularly, FIG. 5 depicts utilizing a kit defining a plurality of separate compartments, each configured to receive a stoma cleaning accessory. For instance, the method (200) may be utilized with the kit 80 described in reference to FIGS. 2-5 and/or to clean a medical device such as the feeding tube 50 of FIG. 1. It should be recognized that, in other embodiments, the method (200) may be utilized with any configuration of the kit 80 and/or with other medical devices or other configurations of the feeding tube 50. Further, the method (200) may include more or less steps. It should also be recognized that the method (200) may mirror the order and flow of the instructions 140 and the associated markers 142 included in the instructions 140 and used to designate the compartments 100, 114 and pouch 116.

The method (200) may include (202) retrieving gloves from a first compartment and applying the gloves to the hands of a user. The method (200) may further include (204) retrieving a drape from a second compartment and positioning the drape as a working surface. Another step may include (206) retrieving a cleaning solution from a third compartment and applying the cleaning solution to the stoma and/or areas of the patient's skin surrounding the stoma. One step of the method (200) may include (208) retrieving a wipe from a fourth compartment and using the wipe to clean and/or dry the stoma and/or the areas of the patient's skin surrounding the stoma utilizing the cleaning solution. The method (200) may additionally include (210) retrieving a swab from a fifth compartment and using the swab to clean and/or dry the stoma and/or the areas of the patient's skin surrounding the stoma. A further step of the method (200) may include (212) retrieving a stoma pad from a sixth compartment and applying the stoma pad to a medical device operable with the stoma. Though steps 206-210 have been described generally in regards to cleaning the stoma itself, it should be recognized that steps of the method (200) may be utilized to clean the medical device, such as the feeding tube 50, in generally the same manner the stoma cleaning accessories 86 are utilized to clean the stoma.

In one embodiment, the method (200) may further include (214) marking the completion of at least one step of the method on a compliance check-off functionality structure. For example, the user may mark the completion of each step of the method. It should be further understood that the method (200) may further include any of the additional features as described herein.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

What is claimed is:

1. A kit for cleaning a stoma, the kit comprising:
a container comprising a plurality of exterior surfaces housing a plurality of dividers such that the plurality of dividers defines separate compartments within the container, wherein the plurality of exterior surfaces defines an opening such that the separate compartments are accessible by a user of the kit;
a plurality of stoma cleaning accessories, wherein each of the separate compartments is configured to receive at least one of the plurality of stoma cleaning accessories; and
a first external access surface operable with at least one of the exterior surfaces such that the first external access surface selectably allows access to the separate compartments,
wherein at least one of the exterior surfaces or the first external access surface includes instructions and an indication of an order that each of the plurality of cleaning accessories is utilized to clean the stoma, and wherein each of the plurality of stoma cleaning accessories is sequentially utilized to clean the stoma.

2. The kit of claim 1, wherein the first external access surface comprises a first hinged lid.

3. The kit of claim 2, wherein the first hinged lid includes a pouch on an outward face of the first hinged lid defining a cavity configured to receive at least one of the stoma cleaning accessories.

4. The kit of claim 3, wherein the cavity of the pouch is configured to receive at least one drape.

5. The kit of claim 2, wherein the first hinged lid includes a plurality of lid dividers coupled to an inward face of the first hinged lid, wherein the plurality of lid dividers define at least one lid compartment configured to receive at least one of the plurality of stoma cleaning accessories.

6. The kit of claim 1, wherein the kit further comprises:
a second external access surface operable with at least one of the exterior surfaces or the first external access surface such that the second external access surface selectively allows access to the separate compartments.

7. The kit of claim 6, wherein the second external access surface comprises a second hinged lid, wherein the second hinged lid is operable with at least one of the exterior surfaces or the first external access surface to selectively allow access to the separate compartments.

8. The kit of claim 1, wherein each of the exterior surfaces and the first external access surface is light impermeable such that the contents of the container are not externally visible.

9. The kit of claim 1, wherein each of the exterior surfaces and the first external access surface includes a fashionable design such that the purpose of the kit is not apparent.

10. The kit of claim 1, wherein each of the separate compartments is sequentially marked in an order an associated cleaning accessory for each of the separate compartments is utilized to clean the stoma.

11. The kit of claim 10, wherein at least one of the separate compartments sequentially marked includes a marker comprising at least one of an alpha-numerical symbol or color code symbol.

12. The kit of claim 1, wherein at least one of the exterior surfaces or the first external access surface includes a compliance check-off functionality structure such that the user marks a successful use of at least one of the plurality of stoma cleaning accessories.

13. The kit of claim 12, wherein the compliance check-off functionality structure includes at least one of a plurality of stickers or perforated punches to mark the successful use of at least one of the plurality of stoma cleaning accessories.

14. The kit of claim 1, wherein at least one of the plurality of stoma cleaning accessories comprises one or more gloves, drapes, cleaning solutions, wipes operable with the one or more cleaning solutions, swabs, stoma pads, or travel packs.

15. The kit of claim 4, wherein the drape includes instructions and an indication of an order that each of the plurality of cleaning accessories is utilized to clean the stoma.

16. A kit for cleaning a stoma, the kit comprising:
a container comprising a plurality of exterior surfaces housing a plurality of dividers such that the plurality of dividers defines separate compartments within the container, wherein the plurality of exterior surfaces defines an opening such that the separate compartments are accessible by a user of the kit;
a plurality of stoma cleaning accessories, wherein each of the separate compartments is configured to receive at least one of the plurality of stoma cleaning accessories;
a first hinged lid operable with at least one of the exterior surfaces such that the first hinged lid selectably allows access to the separate compartments, wherein the first hinged lid includes a pouch on an outward face of the first hinged lid defining a cavity configured to receive at least one of the stoma cleaning accessories, and wherein the first hinged lid includes a plurality of lid dividers coupled to an inward face of the first hinged lid, wherein the plurality of lid dividers define at least one lid compartment configured to receive at least one of the plurality of stoma cleaning accessories; and
a second hinged lid operable with at least one of the exterior surfaces or the first hinged lid such that the second hinged lid selectively allows access to the separate compartments,
wherein each of the plurality of stoma cleaning accessories is sequentially utilized to clean a stoma.

17. The kit of claim 16, wherein at least one of the exterior surfaces, the first hinged lid, or the second hinged lid includes instructions and an indication of an order that each of the plurality of stoma cleaning accessories is utilized to clean the stoma.

18. The kit of claim 16, wherein at least one of the exterior surfaces, the first hinged lid, or the second hinged lid includes a compliance check-off functionality structure such that the user marks a successful use of at least one of the plurality of stoma cleaning accessories.

19. A method of cleaning a stoma utilizing a kit defining a plurality of separate compartments, each configured to receive a stoma cleaning accessory, the method comprising:
retrieving gloves from a first compartment and applying the gloves to the hands of a user;
retrieving a drape from a second compartment and positioning the drape as a working surface;
retrieving a cleaning solution from a third compartment and applying the cleaning solution to the stoma and/or areas of the patient's skin surrounding the stoma;
retrieving a wipe from a fourth compartment and using the wipe to clean and/or dry the stoma and/or the areas of the patient's skin surrounding the stoma utilizing the cleaning solution;
retrieving a swab from a fifth compartment and using the swab to clean and/or dry the stoma and/or the areas of the patient's skin surrounding the stoma; and
retrieving a stoma pad from a sixth compartment and applying the stoma pad to a medical device operable with the stoma.

20. The method of claim 19, further comprising:
marking the completion of at least one step of the method on a compliance check-off functionality structure.
